(19) United States
(12) Patent Application Publication
(10) Pub. No.: US 2007/0021612 A1
(43) Pub. Date: Jan. 25, 2007

(54) Processes and compounds for preparing histone deacetylase inhibitors and intermediates thereof

(75) Inventors: Paul Helquist, Granger, IN (US); Joakim Lofstedt, Uppsala (SE)

Correspondence Address: JAGTIANI + GUTTAG, 10363-A DEMOCRACY LANE, FAIRFAX, VA, 22030, US

(73) Assignee: University of Notre Dame du Lac, Notre Dame, IN (US)

(21) Appl. No.: 11/529,477

(22) Filed: Sep. 29, 2006

Related U.S. Application Data
(63) Continuation-in-part of application No. 11/108,858, filed on Apr. 19, 2005.
(60) Provisional application No. 60/624,523, filed on Nov. 4, 2004.
(60) Provisional application No. 60/624,524, filed on Nov. 4, 2004.
(60) Provisional application No. 60/656,532, filed on Feb. 28, 2005.

(51) Int. Cl. C07F 9/84; C07F 9/70; C07D 215/12; C07C 259/04

(52) U.S. Cl. 546/2; 556/73; 546/176; 562/621

(57) ABSTRACT

Processes for preparing unsaturated esters useful as intermediates for HDAC inhibitors, by reacting an aldehyde or ketone with compounds having the following formula XX: 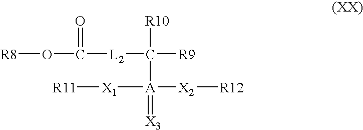
wherein R₈ is an aliphatic group, an aromatic group, or a combined aliphatic and aromatic group; R₉ and R₁₀ are each independently hydrogen, an aliphatic group, an aromatic group, a combined aliphatic and aromatic group, or R₁₀ forms a double bond with L₂ or X₃; R₁₁ and R₁₂ are each independently an aliphatic group, an aromatic group, or a combined aliphatic and aromatic group; L₂ is an aliphatic linking group, an aromatic linking group, or a combined aliphatic and aromatic linking group; A is P or As; L₂ being selected so that the number of carbon atoms directly in the carbon chain between the R₈ and R₉ groups is at least 4, X₁ and X₂ are each independently O or a single bond, and X₃ is O or forms R₁₃ and double bond with R₁₀, wherein R₁₃ is an aliphatic group, an aromatic group, or a combined aliphatic and aromatic group.

### CROSS-REFERENCE TO RELATED APPLICATIONS

This application is a continuation-in-part of U.S. patent application Ser. No. 11/108,858, filed Apr. 19, 2005, which makes reference to and claims the benefit of co-pending U.S. Provisional Patent Applications No. 60/624,523, filed Nov. 4, 2004, Ser. No. 60/624,524, filed Nov. 4, 2004, and Ser. No. 60/656,532, filed Feb. 28, 2005. The entire disclosure and contents of the foregoing Patent Application and Provisional Applications are hereby incorporated by reference herein.

### BACKGROUND

1. Field of the Invention

The present invention relates generally to compounds and processes for preparing histone deacetylase inhibitors, as well as intermediates thereof.

2. Related Art

Histone deacetylase (HDAC) inhibitors are promising compounds for the development of anti-cancer, as well as anti-malarial drugs. HDAC inhibitors include simple fatty acid compounds such as sodium butyrate, phenylbutyrate, and valproic acid, up to more complex cyclic tetrapeptide antibiotics such as apidicin, trapoxin B and depsipeptide. Most of the known HDAC inhibitors are hydroxamic acids or derivatives thereof such as trichostatin A (TSA) and suberoylanilide hydroxamic acid (SAHA). See Wang et al., “QSAR Studies of PC-3 Cell Line Inhibition Activity of TSA and SAHA-Like Hydroxamic Acids,” Bioorg, & Med. Chem. Lett., 14 (2004): pp. 707- 11.

The most potent HDAC inhibitor discovered so far is TSA. TSA is a relatively rare natural product that was originally isolated from Streptomyces hygroscopicus. See Tsji et al., “New Antifungal Antibiotic, Trichostatin,” J. Antibiot., 29 (1976): pp. 1-6; Yoshida et al., “Trichostatin-A and Trapoxin - Novel Chemical Probes for the Role of Histone Acetylation in Chromatin Structure and Function,” BioEssay, 17 (1995): pp. 423-30. TSA has been previously synthesized as both racemic and enantiomerically pure forms. See Fleming et al., “The Total Synthesis of (+/−)-Trichostatin-A—Some Observations on the Acylation and Alkylation of Silyl Enol Ethers, Silyl Dienol Ethers and a Silyl Trienol Ether,” Tetrahedron, 39 (1983): pp. 841-46; K. Mori et al, “Synthetic Microbial Chemistry: Synthesis of Trichostatin-A, a Potent Differentiation Inducer of Friend Leukemic-Cells, and its Antipode,” Tetrahedron, 44 (1988): pp. 6013-20. However, these prior syntheses for TSA require a significant number synthesis steps (e.g., upwards of 20 synthesis steps), and are therefore relatively inefficient and impractical to economically produce TSA. Because these prior syntheses for producing TSA are inefficient, impractical and costly, others have searched for alternative HDAC inhibitors. Indeed, because of the difficulty in synthesizing TSA and the relatively high cost of TSA (e.g., ˜$100/mg.), TSA is used today mainly as a reference substance in the research for new HDAC inhibitors.

Accordingly, it would be desirable to provide a process for synthesizing TSA, including intermediates thereof, as well as other HDAC inhibitors, that: (1) requires fewer synthesis steps; (2) is more practical, (3) is more efficient; (4) is less expensive; and/or (5) has the processing flexibility to prepare a wide variety of HDAC inhibitors, and their respective intermediates, by using the same or similar processing steps.

### SUMMARY

According to a first broad aspect of the present invention, there is provided a process for preparing an unsaturated ester comprising the following steps:
(a) providing an aldehyde or ketone having the following formula I: 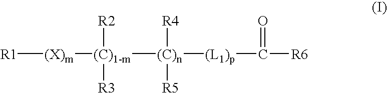
wherein R₁ is an aromatic group or a combined aliphatic and aromatic group; X is —0——S—, —COO—, —OOC—, —CONR₇—, or —R₇NCO—; L₁ an aliphatic linking group, an aromatic linking group, or a combined aliphatic and aromatic linking group; R₂ and R₃ are each independently hydrogen, a hydroxy group, an alkoxy group, an amino group, a carboxy group, an amide group, an ester group, a carbamate group, an aliphatic group, an aromatic group, a combined aliphatic and aromatic group, R₂ and R₃ together are ═O, or one of R₂ and R₃ form a double bond with one of R₄ and R₅; R₄ and R₅ are each independently hydrogen, a hydroxy group, an alkoxy group, an amino group, a carboxy group, an amide group, an ester group, a carbamate group, an aliphatic group, an aromatic group, a combined aliphatic and aromatic group, or one of R₄ and R₅ form a double bond with one of R₂ and R₃; R₆ is hydrogen, an aliphatic group, an aromatic group, or a combined aliphatic and aromatic group; R₇ is hydrogen, an aliphatic group, an aromatic group, or a combined aliphatic and aromatic group; m is 0 or 1; n is 0 or 1; and p is 0 or 1; m, n, p, and L₁ being selected so that the number of carbon atoms directly in the carbon chain between the R₁ and R₆ groups is at least 2;
(b) providing an ester having the following formula II: 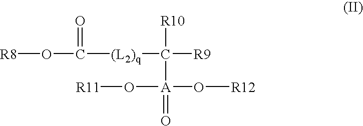
wherein R₈ is an aliphatic group, an aromatic group, or a combined aliphatic and aromatic group; R₉ and R₁₀ are each independently hydrogen, an aliphatic group, an aromatic group, a combined aliphatic and aromatic group, or one of R₉ and R₁₀ form a double bond with L₂; R₁₁, and R₁₂ are each independently an aliphatic group, an aromatic group, or a combined aliphatic and aromatic group; L₂ is an aliphatic linking group, an aromatic linking group, or a combined aliphatic and aromatic linking group; A is P or As; and q is 0 or 1; q and L₂ being selected so that the number of carbon atoms directly in the carbon chain between the R₈ and R₉ groups is at least 2; and
(c) reacting the ester of formula II with the aldehyde or ketone of formula I to form an unsaturated ester having the following formula III: 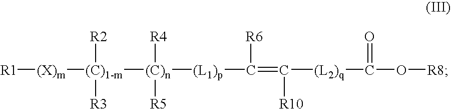
wherein m, n, p, q, L₁ and L₂ are selected so that the number of carbon atoms directly in the carbon chain between the R₁ and R₈ groups is at least 4.

According to a second broad aspect of the invention, there is provided a process for preparing an ester derivative comprising the following steps:
(a) preparing an aldehyde or ketone having the following formula I: 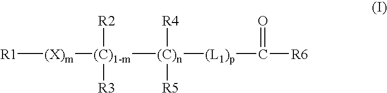
wherein R₁, R₂, R₃, R₄, R₅, R₆, X, L₁, m, n and p are defined as before;
(b) providing an ester having the following formula II: 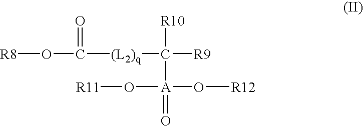
wherein R₈, R₉, R₁₀, R₁₁, R₁₂, A, L₂, and q are defined as before;
(c) reacting the ester of formula II with the aldehyde or ketone of formula I to form an unsaturated ester having the following formula III: 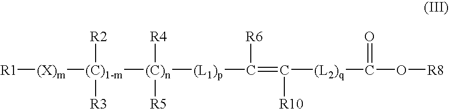
wherein m, n, p, q, L₁ and L₂ are selected so that the number of carbon atoms directly in the carbon chain between the R₁ and R₈ groups is at least 4; and
(d) converting the unsaturated ester of formula III to a derivative having the following formula IV: 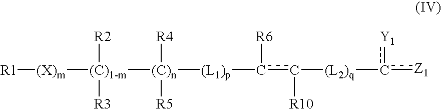
wherein Y₁, is ═O, ═S, ═NR₁₃, —R₁₃ when Z₁, is ═NOR₁₅, or together with Z₁ is ≡N; Z₁, is —OH, halo, —R₁₃, —NR₁₃R₁₄, —NR₁₃OR₁₅, —NR₁₃NR₁₃R₁₅, —L₃—NR₁₃R₁₄, —L₃—NR₁₃C(═NR₁₃)NR₁₃R₁₅, —L₃—Y₂R₁₃, —L₃—C(═Y₂)Z₂, —L₃—PO₃R₁₃R₁₅, ═NOR₁₅ when Y₁ is —R₁₃, or together with Y₁ is ═N, wherein L₃ is an aliphatic linking group, an aromatic linking group, or a combined aliphatic and aromatic linking group, R₁₃ and R₁₅ are each independently hydrogen, an aliphatic group, an aromatic group or a combined aliphatic and aromatic group, R₁₄ is hydrogen, a hydroxy group, an aliphatic group, an aromatic group or a combined aliphatic and aromatic group, Y₂ is O or S, and Z₂ is —Y₂R₁₃, —NR₁₃R₁₄, or —NR₁₃NR₁₃R₁₅.

According to a third broad aspect of the invention, there is provided a process for preparing trichostatic acid or trichostatin A comprising the following steps:
(a) providing an aldehyde having the following formula V: 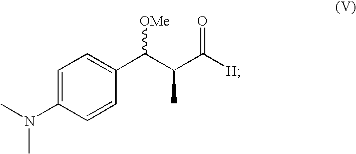
(b) providing a unsaturated phosphonate ester having the following formula VI: 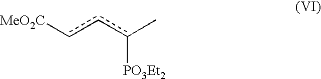
(c) reacting the aldehyde of formula V with the unsaturated phosphonate ester of formula VI to provide an unsaturated ester having the following formula VII: 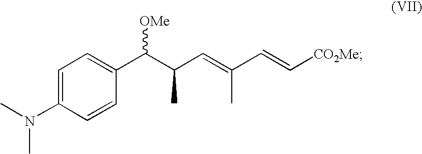
(d) hydrolyzing the unsaturated ester of formula VII to provide a carboxylic acid having the following formula VIII: 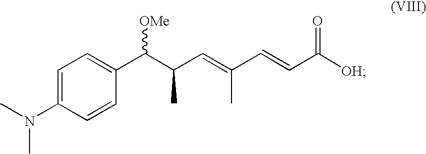
(e) converting the carboxylic acid of formula VIII to trichostatic acid; and
(f) optionally converting the trichostatic acid of step (e) to trichostatin A.

According to a fourth broad aspect of the present invention, there is provided a compound having the following formula XX: 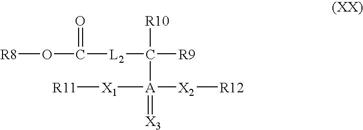
wherein R₈ is an aliphatic group, an aromatic group, or a combined aliphatic and aromatic group; R₉ and R₁₀ are each independently hydrogen, an aliphatic group, an aromatic group, a combined aliphatic and aromatic group, or R₁₀ forms a double bond with L₂ or X₃; R₁₁, and R₁₂ are each independently an aliphatic group, an aromatic group, or a combined aliphatic and aromatic group; L₂ is an aliphatic linking group, an aromatic linking group, or a combined aliphatic and aromatic linking group; A is P or As; L₂ being selected so that the number of carbon atoms directly in the carbon chain between the R₈ and R₉ groups is at least 4, X₁ and X₂ are each independently O or a single bond, and X₃ is O or forms R₁₃ and double bond with R₁₀, wherein R₁₃ is an aliphatic group, an aromatic group, or a combined aliphatic and aromatic group.

According to a fifth broad aspect of the present invention, there is provided a process for preparing an unsaturated ester comprising the following steps:
(a) providing an aldehyde or ketone having the following formula I: 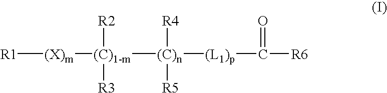
wherein R₁ is an aromatic group or a combined aliphatic and aromatic group; X is —O—, —S—, —COO—, —OOC—, —CONR₇—, or —R₇NCO—; L₁ an aliphatic linking group, an aromatic linking group, or a combined aliphatic and aromatic linking group; R₂ and R₃ are each independently hydrogen, a hydroxy group, an alkoxy group, an amino group, a carboxy group, an amide group, an ester group, a carbamate group, an aliphatic group, an aromatic group, a combined aliphatic and aromatic group, R₂ and R₃ together are ═O, or one of R₂ and R₃ form a double bond with one of R₄ and R₅; R₄ and R₅ are each independently hydrogen, a hydroxy group, an alkoxy group, an amino group, a carboxy group, an amide group, an ester group, a carbamate group, an aliphatic group, an aromatic group, a combined aliphatic and aromatic group, or one of R₄ and R₅ form a double bond with one of R₂ and R₃; R₆ is hydrogen, an aliphatic group, an aromatic group, or a combined aliphatic and aromatic group; R₇ is hydrogen, an aliphatic group, an aromatic group, or a combined aliphatic and aromatic group; m is 0 or 1; n is 0 or 1; and p is 0 or 1; m, n, p, and L₁ being selected so that the number of carbon atoms directly in the carbon chain between the R₁ and R₆ groups is at least 2;
(b) providing an ester having the following formula XXI: 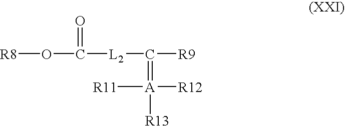
wherein R₈ is an aliphatic group, an aromatic group, or a combined aliphatic and aromatic group; R₉ is hydrogen, an aliphatic group, an aromatic group, or a combined aliphatic and aromatic group; R¹¹, R₁₂ and R₁₃ are each independently an aliphatic group, an aromatic group, or a combined aliphatic and aromatic group; L₂ is an aliphatic linking group, an aromatic linking group, or a combined aliphatic and aromatic linking group; A is P or As; L₂ being selected so that the number of carbon atoms directly in the carbon chain between the R₈ and R₉ groups is at least 4; and
(c) reacting the ester of formula XXI with the aldehyde or ketone of formula I to form an unsaturated ester having the following formula III: 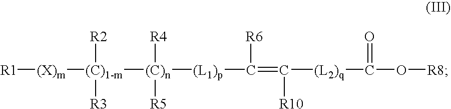
wherein m, n, p, q, L₁ and L₂ are selected so that the number of carbon atoms directly in the carbon chain between the R_{1 and R}₈ groups is at least 4.

According to a sixth broad aspect of the invention, there is provided a process for preparing trichostatic acid or trichostatin A comprising the following steps:
(a) providing an aldehyde having the following formula V: 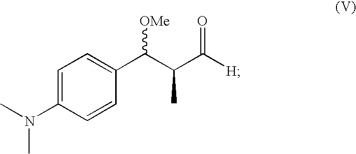
(d) providing a unsaturated ester having the following formula XXII: 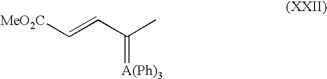
   wherein A is P or As;
(e) reacting the aldehyde of formula V with the unsaturated ester of formula XXII to provide an unsaturated ester having the following formula VII: 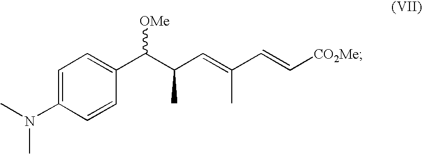
(g) hydrolyzing the unsaturated ester of formula VII to provide a carboxylic acid having the following formula VIII: 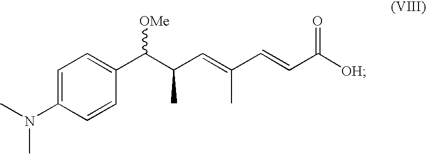
(h) converting the carboxylic acid of formula VIII to trichostatic acid; and
(i) optionally converting the trichostatic acid of step (e) to trichostatin A.

Embodiments of the present invention provide an efficient, practical and cost effective process to prepare unsaturated esters of formula III that are useful as intermediates in preparing, for example, HDAC inhibitors, and potentially HDAC inhibitor prodrugs. Certain embodiments of the present invention especially provide a more, efficient, practical and cost effective process to prepare HDAC inhibitors, such as trichostatin A, and especially intermediates such as trichostatic acid, that can be readily converted into trichostatin A or other HDAC inhibitors/prodrugs. The present invention also provides the flexibility to prepare a wide of variety of HDAC inhibitors/prodrugs, and respective intermediates thereof, using the same or similar processing steps. Embodiments of the present invention also generally require fewer processing steps to obtain the desired intermediates and HDAC inhibitors/prodrugs, including trichostatic acid and trichostatin A.

### BRIEF DESCRIPTION OF THE DRAWINGS

The invention will be described in conjunction with the accompanying drawings, in which:

FIG. 1 illustrates Scheme 1 of the process of the present invention for preparing trichostatin acid and trichostatin A; and

FIGS. 2 and 3 show some representative HDAC inhibitors other than trichostatin A that may be prepared by the process of the present invention.

### DETAILED DESCRIPTION

It is advantageous to define several terms before describing the invention. It should be appreciated that the following definitions are used throughout this application.

Definitions

Where the definition of terms departs from the commonly used meaning of the term, applicant intends to utilize the definitions provided below, unless specifically indicated.

For the purposes of the present invention, the term “aromatic” refers to an unsaturated cyclic arene moiety containing one or more unsaturated cyclic rings (typically 5 and/or 6 atoms per ring) that can be substituted, unsubstituted, or a combination thereof, can be heterocyclic (i.e., including one or more oxygen atoms, nitrogen atoms, sulfur atoms, etc.), nonheterocyclic, or a combination thereof, can have any desired number of carbon atoms, e.g., from 3 to 30 carbon atoms, typically from 3 to 18 carbon atoms, more typically from 3 to 12 carbon atoms, etc. Aromatic moieties suitable herein can include, but are not limited to, substituted or unsubstituted phenyl, naphthyl, biphenyl, binaphthyl, phenanthenryl, anthracenyl, pyridinyl, pyrimidinyl, purinyl, pyrinyl, furanyl, thiophenyl, benzofuranyl, benzothiophenyl, dibenzofuranyl, dibenzothiophenyl, imadazolyl, oxazolyl, thiazolyl, pyrazolinyl, indolyl, pyridazinyl, pyrazinyl, triazolyl, quinolinyl, isoquinolinyl, benzoquinolinyl, phenanthrolinyl (e.g., 1,10-phenanthrolyl), carbazolyl, etc. Suitable substituents can include, but are not limited to, halo (i.e., fluoro, chloro, bromo, iodo), alkyl (e.g., methyl, ethyl, propyl, butyl, etc.) and substituted alkyl (e.g., hydroxymethyl, hydroxyethyl, trifluoromethyl, alkoxymethyl, etc.), amino and substituted amino (e.g., dimethylamino, etc.), hydroxy (e.g., a phenol), carboxy, sulfonate, ester, amide, sulfonamide, carbamate, acyl (i.e., aldehyde or ketone), nitro, etc., or any combination thereof.

For the purposes of the present invention, the term “aliphatic” refers to a carbon-containing moiety other than an aromatic moiety. Aliphatic moieties can be straight chain, branched chain, cyclic (cycloaliphatic), or any combination thereof, can be substituted or unsubstituted, can include one or more heteroatoms (e.g., oxygen atoms, nitrogen atoms, sulfur atoms, etc.) in the carbon chain (i.e., can be heterocyclic), can be unsaturated (i.e., one, two or more double bonds) or saturated, etc, and can have any desired number of carbon atoms, e.g., from 1 to 30 carbon atoms, typically from 1 to 12 carbon atoms, more typically from 1 to 6 carbon atoms, etc. Aliphatic moieties suitable herein can include, but are not limited to, substituted or unsubstituted alkyl, alkenyl, alkadienyl, alkynyl, cycloalkyl, cycloalkenyl, etc. Suitable substituents can include, but are not limited to, halo (i.e., fluoro, chloro, bromo, iodo), alkyl (e.g., methyl, ethyl, propyl, butyl, etc.) and substituted alkyl (e.g., hydroxylmethyl, hydroxyethyl, trifluoromethyl, alkoxymethyl, etc.), hydroxy, amino and substituted amino (e.g., dimethylamino, etc.), carboxy, sulfonate, ester, amide, sulfonamide, carbamate, acyl (i.e., aldehyde or keto), etc., or any combination thereof.

For the purposes of the present invention, the term “combined aliphatic and aromatic” refers to a moiety comprising one or more aliphatic moieties and one or more aromatic moieties. Suitable combined aliphatic and aromatic moieties can include, but are not limited to, unsubstituted and substituted benzyl, phenylethyl, phenylpropyl, phenylbutyl, tribenzylmethyl, tribenzylethyl, phenylalkenyl, phenylalkadienyl, phenylalkatrienyl, phenylalkynl, etc., or any combination thereof.

For the purposes of the present invention, the formulas used in the specification, in the claims or in the drawings can represent a single compound, a mixture of compounds, a single enantiomer or a mixture of enantiomers (i.e., a racemic mixture), a single diastereomer or a mixture of diastereomers, etc., unless otherwise specified.

For the purposes of the present invention, the bond symbol “---” used in the formulas in the specification, in the claims or in the drawing figures represents a bond that can be either a single or double bond, unless otherwise specified.

For the purposes of the present invention, the following abbreviations are used in the specification, in the claims or in the drawing figures: “Bn” refers to benzyl; “Me” refers to methyl; “Et” refers to ethyl; “Bu₂BOTf” refers to di-n-butyl borontrifluoromethanesulfonate; “Et₃N” refers to triethylamine; “TFA” refers to trifluoroacetic acid; “MeOH” refers to methanol; “LiBH₄” refers to lithium borohydride; “S0₃” refers to sulfur trioxide; “LiHMDS” refers to lithium hexamethyldisilazide; “LiOH” refers to lithium hydroxide; “EtOCOCI” refers to ethyl chloroformate.

For the purposes of the present invention, the term “HDAC” is used to refer to histone deacetylases. Histone deacetylases are enzymes that typically regulate the hydrolysis of the ε-acetylated lysine in histones. See Wang et al., “QSAR Studies of PC-3 Cell Line Inhibition Activity of TSA and SAHA-Like Hydroxamic Acids,” Bioorg, & Med. Chem. Lett., 14 (2004): pp. 707-l1 , which is incorporated by reference.

For the purposes of the present invention, the term “HDAC inhibitor” refers to those compounds, compositions, molecules, etc., that partially or completely inhibit the activity of histone deacetylases, including prodrugs.

For the purposes of the present invention, the term “prodrug” refers to a compound, molecule, etc., that is converted by metabolic processes in the body into an active form of a drug, e.g., an HDAC inhibitor.

For the purposes of the present invention, the term “intermediate” typically refers to a compound or compounds that are prepared by a process or step of the present invention that is a precursor of, and can be subsequently used, directly or indirectly, to prepare an end product. For example, intermediates can be used to prepare other intermediates that are then used to prepare an end product. In certain instances, intermediates that are prepared by the process of the present invention can also function as prodrugs.

For the purposes of the present invention, the term “end product” refers to the product obtained at the end or completion of the process, and is typically the product that is ultimately desired from the process.

For the purposes of the present invention, the term “process” refers to one or more steps used to prepare one or more compounds, including one or more intermediates, as well as one or more end products.

For the purposes of the present invention, the term “scheme” refers to a synthesis design, framework, etc., comprising two or more processing steps for preparing specific intermediates and/or end products.

Description

Aspects of the present invention are generally directed to compounds and processes for preparing certain unsaturated esters useful as intermediates in preparing various end products, and in particular HDAC inhibitors and/or HDAC inhibitor prodrugs, including trichostatic acid and trichostatin A. The process of embodiments of the present invention can encompass the processing step or steps for preparing the unsaturated esters, including any processing steps required for synthesizing, preparing or providing reactants used in preparing these unsaturated esters, as well as any subsequent processing steps for converting these unsaturated esters into other compounds, including conversion into intermediates used in preparing HDAC inhibitors, conversion into HDAC inhibitors, and/or conversion into HDAC inhibitor prodrugs.

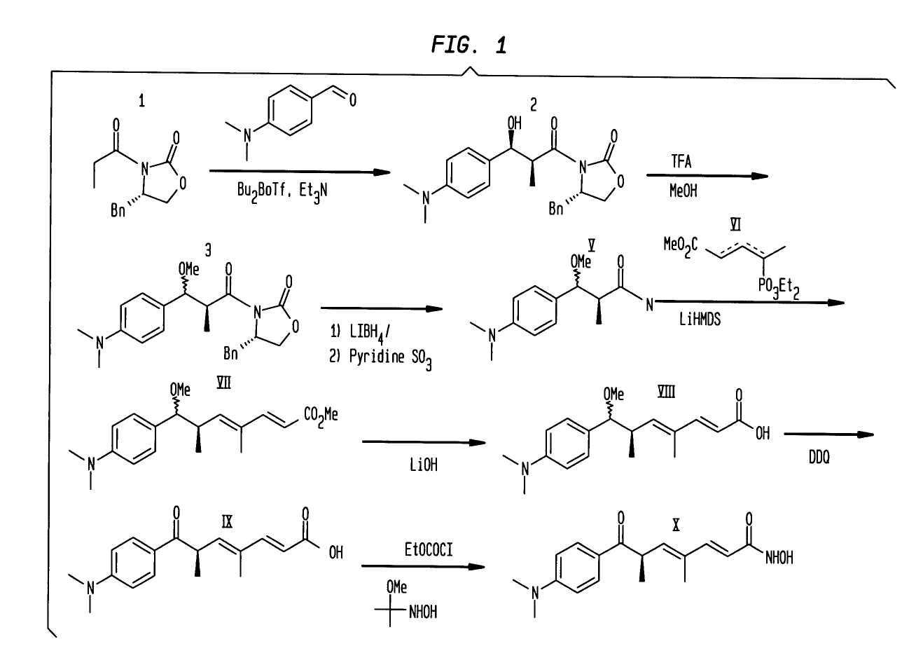

The unsaturated esters that are prepared by certain processes of the present invention have the following formula III: 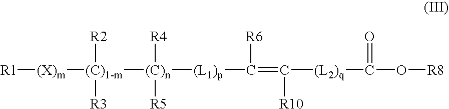
wherein R₁, R₂, R₃, R₄, R₅, R₆, R₈, R₁₀, X, L₁ and L₂ are defined hereafter. For example, in the unsaturated ester of formula III that is typically the intermediate used in preparing trichostatic acid (as well as trichostatin A), R₁ is a 4-dimethylaminophenyl group, m is 0, n is 1, p is 0, R₂ is an alkoxy group having from 1 to 4 carbon atoms, and is typically methoxy, R₃ is hydrogen, R₄ is hydrogen, R₅ is methyl, R₆ is hydrogen, R₈ is typically an alkyl group having from 1 to 4 carbon atoms, and more typically methyl, q is 1, L₂ is —CH₂═CH₂—, R₁₀ is methyl, as represented, for example, by the unsaturated ester having formula VII shown in Scheme 1 of FIG. 1.

For the unsaturated esters of formula III (e.g., those of formula VII), the m, n, p and q values, as well as the L₁ and L₂ groups, are selected so that the number of carbon atoms directly in the carbon chain between the R₁ and R₈ groups is at least 4, typically from 4 to 8, more typically from 7 to 8. HDAC inhibitors and intermediates thereof, such as trichostatic acid and trichostatin A, prepared from such unsaturated esters of formula III (e.g., of those formula VII) have at least 4 carbon atoms in this carbon chain, typically from 4 to 8 carbon atoms, and more typically from 7 to 8 carbon atoms.

One of the reactants used in preparing the unsaturated esters of formula III is an aldehyde or ketone having the following formula I: 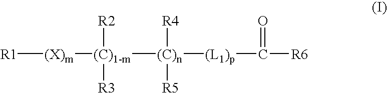
wherein R₁ is an aromatic group or a combined aliphatic and aromatic group; X is —O—, —S—, —COO—, —OOC—, —CONR₇—, or —R₇NCO—; L₁ an aliphatic linking group, an aromatic linking group, or a combined aliphatic and aromatic linking group; R₂ and R₃ are each independently (i.e., same or different) hydrogen, a hydroxy group, an alkoxy group, an amino group, a carboxy group, an amide group (e.g., —OCNHR₁₆, wherein R₁₆ is, for example, an aromatic group or a combined aromatic and aliphatic group), an ester group, a carbamate group (e.g., —NHCOOR₁₆, wherein R₁₆ is, for example, an aromatic group or a combined aromatic and aliphatic group), an aliphatic group, an aromatic group, a combined aliphatic and aromatic group, R₂ and R₃ together are ═O, or one of R₂ and R₃ form a double bond with one of R₄ and R₅; R₄ and R₅ are each independently (i.e., same or different) hydrogen, a hydroxy group, an alkoxy group, an amino group, a carboxy group, an amide group, an ester group, a carbamate group, an aliphatic group, an aromatic group, a combined aliphatic and aromatic group, or one of R₄ and R₅ form a double bond with one of R₂ and R₃; R₆ is hydrogen, an aliphatic group, an aromatic group, or a combined aliphatic and aromatic group; R₇ is hydrogen, an aliphatic group, an aromatic group, or a combined aliphatic and aromatic group; m is 0 or 1; n is 0 or 1; and p is 0 or 1. R_{1, R}₂, R₃, R₄, R₅, R₆, X, L₁, m, n and p are typically selected depending on the unsaturated ester of formula III that is desired, to protect other potentially reactive sites on the aldehyde/ketone of formula I, etc. For example, when the R₁ group is a substituted aromatic group, the substituent is typically an electron donating substituent such as halo (e.g., bromo, etc.), amino or substituted amino (e.g., dimethylamino, etc.). The values for m, n, and p, as well as the L₁ group, are also selected so that the number of carbon atoms directly in the carbon chain between the R₁ and R₆ groups is at least 2, typically from 2 to 5, more typically from 3 to 4. For example, in the aldehyde of formula I that is typically used to prepare the unsaturated ester of formula VII, R₁ is a 4-dimethylaminophenyl group, m is 0, n is 1, p is 0, R₂ is an alkoxy group having from 1 to 4 carbon atoms and is typically methoxy, R₃ is hydrogen, R₄ is hydrogen, R₅ is methyl and R₆ is hydrogen, as represented, for example, by the aldehyde having formula V shown in Scheme 1 of FIG. 1.

Another reactant used in preparing the unsaturated esters of formula III is an ester having the following formula II: 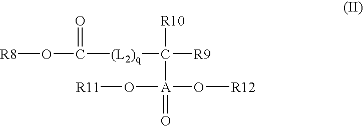
wherein R₈ is an aliphatic group, an aromatic group, or a combined aliphatic and aromatic group; R₉ and R₁₀ are each independently (i.e., same or different) hydrogen, an aliphatic group, an aromatic group, a combined aliphatic and aromatic group, or one of R₉ and R₁₀ form a double bond with L₂; R₁₁ and R₁₂ are each independently (i.e., same or different) an aliphatic group, an aromatic group, or a combined aliphatic and aromatic group; L₂ is an aliphatic linking group, an aromatic linking group, or a combined aliphatic and aromatic linking group; A is P or As; and p is 0 or 1. Typically, A is P, i.e., the esters of formula II are typically phosphonate esters, and R₁ and R₁₂ are each typically an alkyl group having from I to 4 carbon atoms such as ethyl or methyl. R₈, R₉, R₁₀, R₁₁, R₁₂, A, L₂, and q are typically selected depending on the unsaturated ester of formula III that is desired, to favor or promote the desired reaction with the aldehyde/ketone of formula I, etc. The value for q and the L₂ group are also selected so that the number of carbon atoms directly in the carbon chain between the R₈ and R₉ groups is at least 2, typically from 2 to 5, and more typically from 3 to 4. For example, in the ester of formula II that is typically used to prepare the unsaturated ester of formula VIII, R₈ is typically an alkyl group having from 1 to 4 carbon atoms such as methyl, q is 1, L₂ typically has at least one double bond (e.g., —CH₂═CH₂—) and/or forms a double bond with one of R₉ and R₁₀, R₉ is hydrogen or forms a double bond with L₂, R₁₀ is a methyl group, A is P, and R₁₁, and R₁₂ are each typically an alkyl group having from 1 to 4 carbon atoms such as ethyl, as represented, for example, by the unsaturated phosphonate ester having formula VI shown in Scheme 1 of FIG. 1. The unsaturated phosphonate esters such as those of formula VI can be prepared by an Arbuzov reaction using, for example, the respective alkyl halides and trialkyl phosphites as reactants, and typically provide a mixture of unsaturated phosphonate esters (e.g., the 2- and 3-unsaturated phosphonate esters represented in formula VI). See A. Arbuzov et al., J. Russ Phys. Chem. Soc., 46 (1914): p. 295 et. seq., (incorporated by reference herein), for a description of a general approach for providing phosphonate esters of formula III and especially formula VI.

In preparing unsaturated esters of formula III, the ester of formula II is typically reacted with the aldehyde/ketone of formula I under reaction conditions that favor or promote the nucleophilic addition of the carbon adjacent to the A group of the ester of formula II, to the carbonyl carbon adjacent to the R₆ group of the aldehyde/ketone of formula II, while at the same minimizing other undesired reactions or effects, including preserving desired stereoisomeric configurations. This reaction typically uses a strong base that is weakly nucleophilic, for example, sodium hydride or alkali metal amide bases such as lithium hexamethyldisilazide, lithium di-isopropyl amide, lithium isopropylcyclohexyl amide, lithium dicyclohexylamide, sodium hexamethyldisilazide, potassium hexamethyldisilazide, lithium amide, etc. This strong base is typically initially added to the ester of formula II at relatively low temperatures (e.g., as low as about −80° C.), with the reaction product of the strong base and ester of formula II then being reacted with the aldehyde/ketone of formula I. The reaction between the aldehyde/ketone of formula I and the ester of formula II can be carried out in any compatible solvent including, but not limited to, ethers such as diethyl ether, methyl ethyl ether, t-butylmethylether, etc., cyclic ethers such as tetrahydrofuran (THF), dioxane, tetrahydropyran, etc., diethers such as dimethoxyethane, etc., esters such as methyl acetate, ethyl acetate, etc., aromatic solvents such as toluene, etc., amides such as dimethylformamide (DMF), dimethylacetamide, etc., acetonitrile, dimethylsulfoxide (DMSO), etc. After the addition of the strong base, the condensation reaction between the aldehydes/ketones of formula I and esters of formula II can be carried out at temperatures in the range of typically from about −200 to about 75° C., more typically in the range from about 0° to about 30° C. The rate at which the reaction proceeds can vary depending on such factors as the aldehydes/ketones of formula I and esters of formula II that are reacted, the strong bases that are used, etc., but is typically complete in from about 1 to about 24 hours, more typically in from about 1 to about 12 hours, with yields of the unsaturated ester of formula III typically in the range of from about 40 to about 80%, and more typically in the range of from about 60 to about 80%.

Another reactant that may be used in preparing the unsaturated esters of formula III is an ester having the following formula XX: 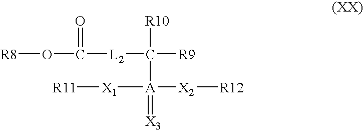
wherein R₈ is an aliphatic group, an aromatic group, or a combined aliphatic and aromatic group; R₉ and R₁₀ are each independently hydrogen, an aliphatic group, an aromatic group, a combined aliphatic and aromatic group, or R₁₀ forms a double bond with L₂ or X₃; R₁₁ and R₁₂ are each independently an aliphatic group, an aromatic group, or a combined aliphatic and aromatic group; L₂ is an aliphatic linking group, an aromatic linking group, or a combined aliphatic and aromatic linking group; A is P or As; L₂ being selected so that the number of carbon atoms directly in the carbon chain between the R₈ and R₉ groups is at least 4, X₁ and X₂ are each independently O or a single bond, and X₃ is O or forms R₁₃ and double bond with R₁₀, wherein R₁₃ is an aliphatic group, an aromatic group, or a combined aliphatic and aromatic group. The variations for the R₈, R₉, R₁₀, R₁₁, R₁₂, A, and L₂ groups of the esters of formula XX may be the same as those for the R₈, R₉, R₁₀, R₁₁, R₁₂, A, and L₂ groups of the ester of formula II. For example, R₈ can be an alkyl group having from 1 to 4 carbon atoms (e.g., a methyl group), R₉ can be hydrogen or an alkyl group having from 1 to 4 carbon atoms, R₁₀ can be hydrogen, an alkyl group having from 1 to 4 carbon atoms or form a double bond with L₂, L₂ can be from 4 to 5 carbon atoms (e.g., 4 carbon atoms) and can be an unsaturated aliphatic linking group or an aliphatic linking group forming a double bond with R₁₀, and R₁₁ and R₁₂ are each an alkyl group having from 1 to 4 carbon atoms (e.g., an ethyl group). When X₁ and X₂ are each a single bond, and when X₃ forms R₁₃ and a double bond with R₁₀, the ester can have the following formula XXI: 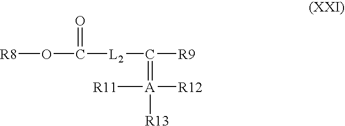
wherein R₈ is an aliphatic group, an aromatic group, or a combined aliphatic and aromatic group; R₉ is hydrogen, an aliphatic group, an aromatic group, or a combined aliphatic and aromatic group; R₁₁, R₁₂ and R₁₃ are each independently an aliphatic group, an aromatic group, or a combined aliphatic and aromatic group; L₂ is an aliphatic linking group, an aromatic linking group, or a combined aliphatic and aromatic linking group; A is P or As; L₂ being selected so that the number of carbon atoms directly in the carbon chain between the R₈ and R₉ groups is at least 4 (e.g., from 4 to 5 carbon atoms, such as 4 carbon atoms). For example, R₁₁, R₁₂ and R₁₃ can each be independently an aromatic group, for example, each can be a phenyl group, as in the ester having the following formula XXII:

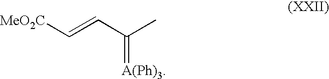

In preparing unsaturated esters of formula III, the ester of formula XX can be reacted with the aldehyde/ketone of formula I under reaction conditions the same or similar to those used for the ester of formula II. When X₁ and X₂ are each a single bond, and when X₃ forms R₁₃ and double bond with R₁₀, (e.g., an ester of formula XXI or XXII), the reaction may also use a base such as a hydroxide, such as, for example, sodium hydroxide, potassium hydroxide, lithium hydroxide, ammonium hydroxide, etc., metal alkoxides, such as, for example, sodium methoxide, potassium methoxide, lithium methoxide, etc., metal dialkyl amides, such as lithium diisopropylamide, sodium bis(trimethylsilyl)amide, potassium bis(trimethylsilyl)amide, etc., metal hydrides such as sodium hydride, potassium hydride, lithium hydride, etc.

The unsaturated esters of formula III can also be converted into derivatives having the following formula IV: 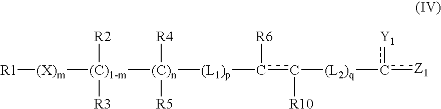
wherein Y₁, is ═O, ═S, ═NR₁₃, —R₁₃ when Z₁, is ═NOR₁₅, or together with Z₁, is ═N; Z₁ is —OH, halo (typically chloro), —R₁₃, —NR₁₃R₁₄, —NR₁₃OR₁₅, —NR₁₃NR₁₃R₁₅, —L₃—NR₁₃R₁₄, —L₃—NR₁₃C(═NR₁₃)NR₁₃R₁₅, —L₃—Y₂R₁₃, —L₃—C(═Y₂)Z₂, —L₃—PO₃R₁₃R₁₅, ═NOR₁₅ when Y₁ is —R₁₃, or together with Y₁, is ═N, wherein L₃ is an aliphatic linking group, an aromatic linking group, or a combined aliphatic and aromatic linking group, typically a saturated or unsaturated aliphatic linking group, and more typically —(CH₂)ₖ₋, wherein k is at least 1, and typically is from 1 to 8, more typically from 1 to 6, R₁₃ and R₁₅ are each independently (i.e., same or different) hydrogen, an aliphatic group (including cyclic or heterocyclic aliphatic groups having heteroatoms such as O, S or N, and typically having from 3 to 6 total atoms in the cyclic ring), an aromatic group or a combined aliphatic and aromatic group, R₁₄ is hydrogen, a hydroxy group, an aliphatic group, an aromatic group or a combined aliphatic and aromatic group, Y₂ is O or S (typically O), and Z₂ is —Y₂R₁₃, —NR₁₃R₁₄, or —NR₁₃NR₁₃R₁₅. Y₁, is typically ═O, while Z₁ is typically a —OH (i.e., a carboxylic acid), —NR₁₃R₁₄ wherein R₁₃ is typically hydrogen and R₁₄ is typically hydroxy (i.e., a hydroxamic acid), or —CH₂—PO₃R₁₃R₁₅, group, wherein R₁₃ and R₁₅ are each typically hydrogen or an alkyl group of from 1 to 4 carbon atoms, more typically ethyl or methyl (i.e., a phosphonate).

One such derivative that unsaturated esters of formula III can be relatively easily converted into are the respective carboxylic acids (i.e., wherein Y₁ is ═O and Z₁ is —OH in formula IV) having the following formula XI: 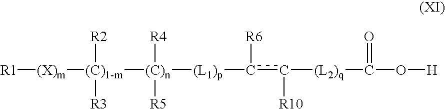
Conversion of the unsaturated esters of formula III into carboxylic acids of formula XI (deesterification) can be achieved by art-recognized hydrolysis reactions, including treatment with a strong base (i.e., saponification) such as lithium hydroxide, sodium hydroxide, potassium hydroxide, etc., to hydrolyze the unsaturated ester of formula III to the respective carboxylic acid of formula XI. For example, as illustrated in Scheme 1 of FIG. 1, the unsaturated ester VII can be hydrolyzed (saponified) by treatment with lithium hydroxide to the respective carboxylic acid VIII.

Carboxylic acids of formula XI can also be converted into various carboxylic derivatives. One such carboxylic acid derivative that can be formed from the carboxylic acid of formula XI is a hydroxamic acid (i.e., wherein Y₁ is ═O and Z₁ is —NHOH in formula IV), as represented by the following formula XII: 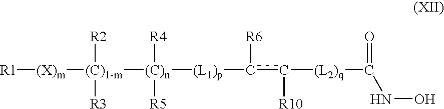
For example, as illustrated in Scheme 1 of FIG. 1, trichostatic acid of formula IX can be reacted (e.g., after activation, for example, by ethyl chloroformate) with a hydroxylamine (e.g., O-(2-methoxy-2-propyl)hydroxylamine) to provide the hydroxamic acid trichostatin A of formula X (i.e., wherein R₁ is 4-dimethylaminophenyl, m is 0, R₂ and R₃ are together ═O, R₄ is hydrogen, R₅ is methyl, p is 0, R₆ is methyl, q is 1, and L₂ is —CH₂═CH₂—in formula XII).

To provide phosphonate derivatives of formula IV (i.e., wherein Y₁ is ═0 and Z₁ is —CH₂—PO₃R₁₃R₁₅), the unsaturated esters of formula III (wherein R₈ is typically a methyl group), or the respective acyl chloride (i.e., wherein R₈ is chloro) are typically reacted with a metalated phosphonate such as a Li—CH₂—PO₃R₁₃R₁₅(wherein R₁₃ and R₁₅ are each typically hydrogen or an alkyl group having from 1 to 4 carbon atoms, and more typically an ethyl or methyl group). See F. Orsini et al., Tetrahedron Lett., 43 (2002): p. 7259 et seq. and references cited therein (herein incorporated by reference), for a description of a general approach for providing these phosphonate derivatives.

The unsaturated esters of formula III, as well as the derivatives of formula IV, can also be subjected to one or more other processing steps. These other processing steps can include the removal of protecting groups, the conversion of existing substituent groups (e.g., alkoxy groups, etc.) to other substituent groups (e.g., ═O, etc.), conversion of double bonds to single bonds (e.g., by hydrogenation) in the carbon chain directly between the R₁ and Z₁ groups (e.g., double bond between the carbon atoms having the R₆ and R₁₀ groups, etc., and can occur prior to and/or after conversion of the unsaturated esters of formula III to the derivatives of formula IV. For example, as illustrated in Scheme 1 of FIG. 1, the carboxylic acid represented by formula VIII that is obtained after deesterification (e.g., saponification) of the unsaturated ester of formula V is typically oxidized by treatment with a selective oxidizing agent such as 2,3-dichloro-5,6-dicyanobenzoquinone (DDQ), 2,3,5,6-tetrachloro-p -benzoquinone (chloranil), etc., to form trichostatic acid (the respective keto acid wherein R₂ and R₃ together become ═O in formula IV), as represented by formula X in Scheme 1 of FIG. 1.

One group of representative ester derivatives that can be obtained by the process of the present invention are those having either of the following formulas XIII or XIV: 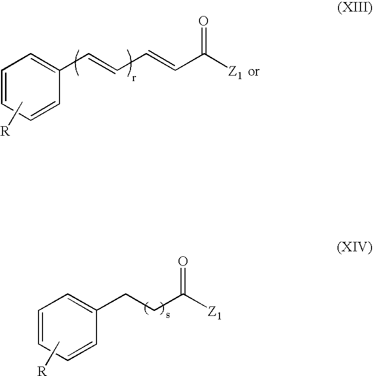
wherein Z₁ is defined as before and is typically —NHOH, or —CH₂—PO₃R₁₃R₁₅, wherein R₁₃ and R₁₅ are each typically hydrogen or an alkyl group having form 1 to 4 carbon atoms, more typically ethyl or methyl; r is at least 1 and is typically form 1 to 3, more typically form 1 to 2; s is at least 2, and is typically from 3 to 7, more typically from 5 to 7; and R is either hydrogen or a substituent group, typically an electron donating substituent group such as halo (e.g., bromo, etc.), amino or substituted amino (e.g., dimethylamino, etc.) that is positioned either ortho (o-) or more typically para (p-) on the benzene ring.

Another group of representative ester derivatives that can be obtained by the process of the present invention are those having either of the following formulas XV or XVI: 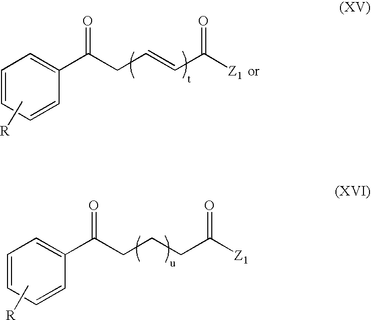
wherein Z₁ is defined as before and is typically —NHOH, or —CH₂—PO₃R₁₃R₁₅, wherein R₁₃ and R₁₅ are each typically hydrogen or an alkyl group having form 1 to 4 carbon atoms, more typically ethyl or methyl; t is at least 1 and is typically form 1 to 3, more typically 1 or 2; u is at least 2 and is typically from 2 to 6, more typically from 2 to 4; and R is either hydrogen or a substituent group, typically an electron donating substituent group such as halo (e.g., bromo, etc.), amino or substituted amino (e.g., dimethylamino, etc.) that is positioned either ortho (o-) or more typically para (p-) on the benzene ring.

Another group of representative ester derivatives that can be obtained by the process of the present invention are those having the following formula XVII: 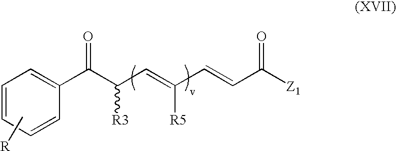
wherein Z₁ is defined as before and is typically —NHOH, or —CH₂—PO₃R₁₃R₁₅, wherein R₁₃ and R₁₅ are each typically hydrogen or an alkyl group having form 1 to 4 carbon atoms, more typically ethyl or methyl; R₃ an alkyl group having form 1 to 2 carbon atoms, typically methyl, R₅ is an alkyl group having form 1 to 4 carbon atoms, typically methyl; v is at least 1, and is typically form 1 to 2, more typically l; and R is either hydrogen or a substituent group, typically an electron donating substituent group such as halo (e.g., bromo, etc.), amino or substituted amino (e.g., dimethylamino, etc.) that is positioned either ortho (o-) or more typically para (p-) on the benzene ring.

A particularly representative carboxylic acid or carboxylic acid derivative of formula XVII that can be obtained by the process of the present invention is trichostatic acid (R is p- or 4-dimethylamino; R₃ and R₅ are each methyl; v is 1; and Z₁ is —OH) or trichostatin A (R is p- or 4-dimethylamino; R₃ and R₅ are each methyl; t is 1 and Z₁ is —NHOH). Scheme 1 of FIG. 1 illustrates an embodiment of a synthesis of trichostatic acid or trichostatin A according to the process of the present invention, starting with the N-acyl oxazolidinone compound of formula 1. See Evans, J. Am. Chem. Soc., 103 (1981), p. 2127 et seq. (herein incorporated by reference), for a description of the preparation of compound 1. As shown in step 1 of Scheme 1, compound 1 is reacted with 4-(dimethylamino)benzaldehyde using, for example, triethylamine and di-n-butylboryl trifluoromethanesulfonate, to provide the respective compound 2 (typically a mixture of diasterioisomers). See Evans, J. Am. Chem. Soc., supra, for a description of the preparation of compound 2. As shown in step 2 of Scheme 1, compound 2 is then reacted with methanol using trifluoroacetic acid as the catalyst to convert the hydroxy group to a methoxy group in providing the respective compound 3. As shown in step 3 of Scheme 1, compound 3 is converted to the aldehyde of formula V in either one or two steps by treatment with lithium borohydride, followed by treatment with a pyridine/sulfur trioxide complex. See Djuric, Tetrahedron Lett., 29 (1988): pp. 3459-62 (herein incorporated by reference), for a description of the lithium borohydride treatment step; Parikh et al., J. Am. Chem. Soc., 89 (1967): p. 5505 et seq. (herein incorporated by reference), for a description of the pyridine/sulfur trioxide treatment step. Typically, compound 3 is converted in step 3 to the aldehyde of formula V just prior to carrying out step 4 of Scheme 1.

As further illustrated in step 4 of Scheme 1 in FIG. 1, the aldehyde of formula V is combined with the reaction product of the unsaturated phosphonate ester of formula VI (typically a mixture of 2- and 3-unsaturated phosphonate esters) and lithium hexamethyldisilazide (as the weakly nucleophilic/strong base) in a solvent such as THF. The reaction temperature is gradually warmed from about −78° C. when the reagents are initially added or combined together, up to about 25° C. to provide the unsaturated ester of formula VII (typically as a 1:2 ratio cis:trans isomers) in yields of from about 60 to about 80%. (Alternatively, an ester of formula XXII wherein, for example, A is P may be used instead of phosphonate ester of formula VI.) In step 5 of Scheme 1, the unsaturated ester of formula VII is hydrolyzed (saponified) by treatment with lithium hydroxide to provide the respective carboxylic acid of formula VIII. See Corey, Tetrahedron Lett., (1977): p. 3529 et seq. (herein incorporated by reference); K. Mori et al., “Synthetic Microbial Chemistry: Synthesis of Trichostatin-A, a Potent Differentiation Inducer of Friend Leukemic-Cells, and its Antipode,” Tetrahedron, 44 (1988): pp. 6013-20 (herein incorporated by reference), for a general description of how to carry out hydrolysis/saponification step 5. In step 6 of Scheme 1, the carboxylic acid of formula VIII is oxidized by DDQ to form trichostatic acid (formula IX). See I. Fleming et al., “The Total Synthesis of (+/−)-Trichostatin-A—Some Observations on the Acylation and Alkylation of Silyl Enol Ethers, Silyl Dienol Ethers and a Silyl Trienol Ether,” Tetrahedron, 39 (1983): pp. 841-46 (herein incorporated by reference), for a general description of how to carry out oxidation step 6 to obtain trichostatic acid. In step 7 of Scheme 1, trichostatic acid is reacted with a hydroxylamine (e.g., O-(2-methoxy-2-propyl)hydroxylamine, O-benzyl-hydroxylamine, O-(4-methoxybenzyl)hydroxylamine, O-(4-nitrobenzyl)-hydroxylamine, O-trimethylsilyl-hydroxylamine, O-(tert-butyldimethylsilyl) -hydroxylamine, O-(tert-butyl)-hydroxylamine, O-(tert-butoxycarbonyl)-hydroxylamine, O-trityl-hydroxylamine, O-tetrahydropyranyl-hydroxylamine, etc.) using ethyl chloroformate to provide the hydroxamic acid trichostatin A (formula X). See K. Mori et al., supra, for a general description of how to carry out step 7 to obtain trichostatin A.

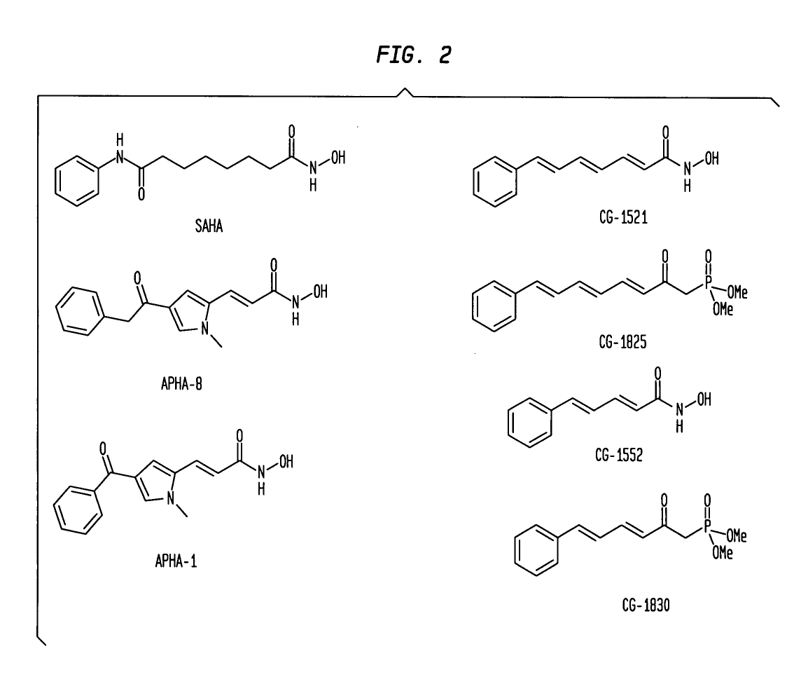

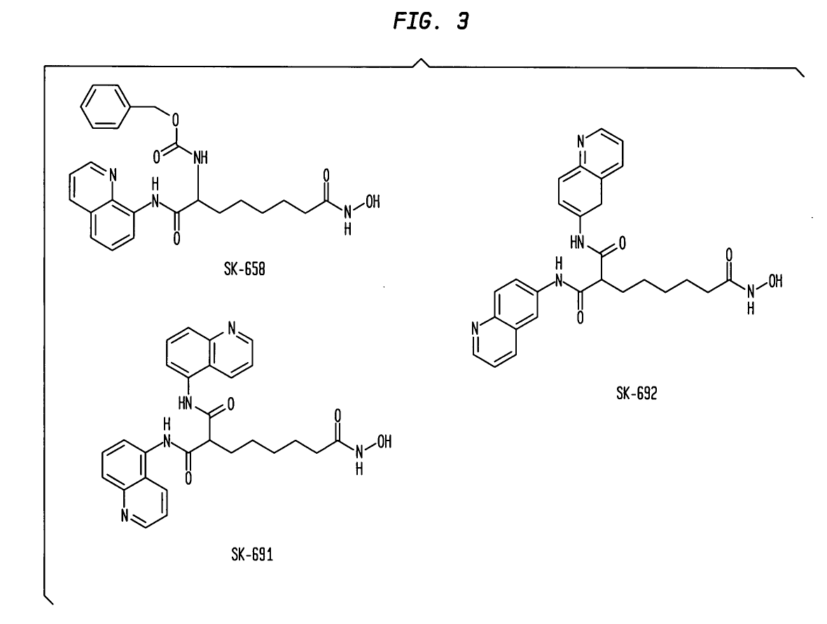

In addition to trichostatic acid and trichostatin A, the process of the present invention can be used to prepare a variety of HDAC inhibitors (e.g., carboxylic acids, hydroxamic acids, other carboxylic acid derivatives, etc.), their respective intermediates, as well as other compounds useful for other pharmacological or drug uses. Other representative HDAC inhibitors that can be prepared by the process of the present invention include those shown in FIGS. 2 and 3 and identified as suberoylanilide hydroxamic acid (SAHA), the diamide or amide-carbamate hydroxamic acids identified as SK-658, SK-692, and SK-691, the unsaturated aliphatic aromatic hydroxamic acids identified as APHA-1 and APHA-8, the unsaturated aliphatic hydroxamic acids identified as CG 1521 and CG 1552, and the unsaturated aliphatic 2-ketophosphonates identified as CG 1825, and CG 1830. See Wang et al., “On the Function of the 14 Å Long Internal Cavity of Histone Deacetylase-Like Protein: Implications for the Design of Histone Deacetylase Inhibitors,” J. Med. Chem., 47 (2004): pp. 3409-17, which is incorporated by reference. The process of the present invention is especially useful in preparing HDAC inhibitors, and intermediates thereof, such as trichostatic acid and trichostatin A, compounds such as CG 1521, CG 1552, CG 1825 and CG 1830, etc., that require the preservation of certain desired stereoisomeric configurations and/or have two or more double bonds directly in the carbon chain between the R₁ and R₈ groups, and in particular where the number of carbon atoms directly in this chain is from 4 to 8, more typically from 7 to 8, as well as those HDAC inhibitors, and intermediates thereof.

### EXAMPLE

The following is a detailed description of the synthesis of trichostatic acid and trichostatin A according to Scheme 1 of FIG. 1:

Step 1: Synthesis of Compound 2.

Di-n-butylboryl trifluoromethanesulfonate (2.35 mL, 2.35 mmol, 1M in DCM) is added dropwise over 1 hour to a 0° C. solution of compound 1 ((S)-3-(1-oxoprop-1-yl)-4-(phenylmethyl-1,3-oxazolidin-2-one) (0.5 g, 2.14 mmol) in 2 mL of DCM. To the resulting copper colored solution is added triethylamine (0.361 mL, 2.6 mmol) over 0.5 hours. The resulting yellow solution is cooled to −78° C., a solution of 4-(dimethylamino)benzaldehyde (356 mg, 2.35 mmol) in 2 mL of dichloromethane (DCM) is then added dropwise, and the temperature is maintained at −78° C. for 20 min. This mixture is allowed to warm to 0° C., and is then stirred at 0° C. for 1 hour. The tan solution is cooled to −10° C., and the reaction is then quenched by the addition of a pH 7 phosphate buffer solution (2.15 mL). A solution of MeOH:30% aqueous H₂O₂ (2:1 ratio, 6.5 mL) is then added dropwise while keeping the temperature below 10° C., and the resulting mixture is then stirred at 0° C. for 1 hour. Volatiles are then removed by evaporation under vacuum, and the residue is extracted with DCM. The combined organic extracts are washed with saturated aqueous NaHCO₃ and brine, dried over MgSO₄, filtered, and the volatiles then removed by evaporation under vacuum. The residue is purified by flash chromatography (30% ethyl acetate in hexane) to provide a white solid (0.76 g, 93% yield) comprising compound 2 ((4S)-4-benzyl-3-(2S,3S)-3-[4-(dimethylamino)phenyl]-3-hydroxy-2-methylpropanoyl-1,3-oxazolan-2-one) as a 5:1 mixture of diastereomers. Analytical: IR 3514, 2919, 1779, 1695, 1523, 1384, 1351, 1209, 816, 704 cm⁻¹; ¹H NMR (CDCl₃) 7.36-7.16 (7H, m, ArH's), 6.72-6.66 (2H, d, J=9.0 Hz, ArH's), 4.99-4.91 (1H, d, J=5.1 Hz, CHOH), 4.56-4.46 (1H, m, CHMe), 4.17-4.06 (2H, m, ½ oxazolidinone-CH₂+CHBn), 4.04-3.93 (1H, m, ½oxazolidinone-CH₂), 3.27-3.19+2.79-2.70 (2H, ABX, J=3.0, 13.5, 9.0, 151.8, PhCH₂), 2.92 (6H, s, NMe_{2),} 1.30-1.26 (3H, d, J=6.9 Hz, CHMe); ¹³C NMR (CDCl₃) δ176.77, 153.24, 150.36, 135.45, 129.72, 129.65, 127.64, 127.57, 127.31, 112.61, 74.55, 66.34, 55.61, 44.99, 40.96, 38.04, 12.02; m/z (+veFAB) 382 (M+H); HRMS for C₂₂H₂₆N₂O₄+H calcd 382.1893, found 382.1884.

Step 2: Synthesis of Compound 3.

To a solution of compound 2 from step 1 (2.5 g, 6.5 mmol) in MeOH (250 mL) is dropwise added trifluoroacetic acid until the pH of the mixture reaches 3 to 4. The mixture is then stirred overnight at room temperature. Saturated aqueous NaHCO₃ (50 mL) is then added, the solvent is removed by evaporation under vacuum, and the residue is purified by flash chromatography to provide a white solid (2.0 g, 80% yield) comprising compound 3 ((4S)-4-benzyl-3-(2S,3R/S)-3- [4-(dimethylamino)phenyl]-3-methoxy-2-methylpropanoyl-1,3-oxazolan-2-one) as a 4:1 mixture of diastereomers. Analytical: IR 2980, 2933, 2881, 1781, 1698, 1614, 1523, 1384, 1350, 1210, 1096, 819, 704 cm⁻¹; ¹H NMR (CDC₁₃) δ (major isomer) 7.38-7.14 (7 H, m, ArH's), 6.76-6.70 (2 H, d, J=8.7 Hz, ArH's), 4.82-4.72 (1 H, m, CHOMe), 4.30-4.15 (4 H, m, CH's), 3.35-3.27+2.86-2.76 (2 H, ABX, J=147.3, 13.8, 9.6, 2.7 Hz, CH₂Ph), 3.10 (3 H, s, OMe), 2.98 (6 H, s, NMe₂), 0.98-0.91 (3 H, d, J=6.6 Hz, CHMe); ¹³C NMR (CDCl₃) δ 176.41, 153.53, 150.75, 135.73, 129.77, 129.17, 129.13, 128.57, 127.51, 112.48, 86.05, 66.30, 56.65, 55.81, 44.31, 40.81, 38.25, 14.85; m/z (+veFAB) 396 (M⁺); HRMS for C₂₃H₂₈N₂O₄ calcd 396.2049, found 396.2044.

Step 3: Providing Aldehyde of Formula V.

A solution of compound 3 from step 2 (300 mg, 0.75 mmol) in THF (1 mL) is cooled to 0° C. Methanol (0.053 mL) and LiBH₄ (1.125 mL, 2.25 mmol, 2 M in THF) are added sequentially and the mixture is then stirred at room temperature overnight. The reaction is then quenched by the addition of a 0.5 M solution of Rochelle's salt (2 mL), diluted with ether (2 mL) and then stirred vigorously for 2 hours. The mixture is then separated and extracted further with ether. The combined organic extracts are washed with brine, dried over sodium sulfate, filtered, and the solvent then removed by distillation under vacuum. Purification by column chromatography provides a clear oil (129 mg, 77% yield) comprising a mixture of isomers of the respective alcohol (2R,3R/S)-3-[4-(dimethylamino)phenyl]-3-methoxy-2-methylpropan-1-ol). Analytical: ¹H NMR (CDCl₃) δ (major isomer) 7.18-7.10+6.76-6.67 (4H, AB, J=8.7 Hz, ArH's), 3.92-3.84 (1H, d, J=9.3 Hz, CHOMe), 3.70-3.62 (2H, m, CH₂OH), 3.15 (3H, s, OMe), 2.96 (6H, s, NMe₂), 2.12-2.00 (1H, m, CHMe), 0.65-0.59 (3H, d, J=6.9 Hz, CHMe); ¹³C NMR (CDCl₃) δ (major isomer) 150.52, 128.62, 128.40, 128.13, 112.45, 90.98, 68.77, 56.37, 41.98, 40.82, 14.17. To a solution of this alcohol (200 mg, 0.90 mmol) and triethylamine (0.80 mL, 5.65 mmol) in dry DMSO (1.9 mL) is added a pyridine-sulfur trioxide complex (429 mg, 2.7 mmol) in DMSO (1.9 mL). After the addition of the pyridine-sulfur trioxide complex, the solution is stirred at room temperature for 15 min., poured onto an ice/water mixture, and then extracted with ether. The ether layers are washed with water and then brine, dried over sodium sulfate, filtered, and the solvent then removed by distillation under vacuum (at a temperature below 40° C.). After being left under high vacuum for 2 hours, a product is obtained (in >99% yield) comprising the aldehyde of formula V.

Step 4: Synthesis of Unsaturated Ester (Formula VII).

a. Synthesis of Phosphonate Esters (Formula VI).

To a solution of (E)-4-bromo-2-pentenoic acid (5 g, 28 mmol) in methanol (250 mL) is added a catalytic amount of sulfuric acid. The mixture is stirred at room temperature for 24 hours. Saturated aqueous NaHCO₃ (50 mL) is added, and methanol is then removed by distillation under vacuum. The resulting residue is extracted with ether, and the organic layers are then combined, washed with brine, dried over MgSO₄, filtered, and the solvent then removed by distillation under vacuum to yield a product (80%) comprising methyl (E)-4-bromo-2-pentenoate. This methyl ester (1 g, 5.2 mmol) is heated to 120° C., triethyl phosphite (0.99 mL, 5.6 mmol) is then added dropwise over a 1 hour period, and the mixture held at 120° C. for 24 to 36 hours (while being monitored using NMR samples). The resulting crude mixture is purified by chromatography (using 70% ethyl acetate in hexane), and then distilled to provide a clear oil (0.468 g, 36% yield) comprising a 1:1 mixture of the unsaturated phosphonate ester isomers of formula VI (methyl (E)-4-(diethoxyphosphoryl)-2-pentenoate and methyl (E)4-(diethoxyphosphoryl)-3-pentenoate). Analytical: IR 2984, 1739 (d), 1648, 1438, 1250, 1052, 1023, 965, 791 cm ^{-1;} ¹H NMR (CDCl₃) δ (both isomers) 7.02-6.88 (0.5H, m), 6.75-6.57 (0.5H, dtt, J=23.1, 14.1, 3.3 Hz), 5.96-5.86 (0.5H, ddt, J=18.6, 4.8, 1.5 Hz), 4.15-3.99 (4H, m, OEt), 3.74-3.71 (1.5H, d, J=1.2 Hz, OMe), 3.70-3.67 (1.5H, d. J=1.5 Hz, OMe), 3.25-3.15 (1H, m), 2.70-2.90 (0.5H, m), 1.85-1.76 (0.5H, d, J=14.4 Hz), 1.39-1.26 (4.5H, m); m/z (+veFAB) 251 (M+H⁺); HRMS for C₁₀H₁₉O₅P+H calcd 251.1048, found 251.1045.

b. Reacting Aldehyde (Formula V) with Phosphonate Esters (Formula VI).

To a solution of the mixed unsaturated phosphonate esters of formula VI from step 4a (0.27 g, 1.08 mmol) in THF (4 mL) at −78° C. is added dropwise LiHMDS (1.17 mL, 1.17 mmol, 1M solution in THF). The reaction mixture is stirred at −78° C. for 30 min., warmed to −40° C. over 1 hour, and then cooled back down to −78° C. A solution of the aldehyde of formula V from step 3 in THF (4 mL) is then added to this reaction mixture, and the resulting combined reaction mixture is allowed to reach room temperature while being stirred and left for 24 hours. The stirred mixture is then cooled to 0° C., quenched with saturated aqueous NH₄Cl, and then stirred for 0.5 hours. This stirred mixture is then extracted with ether, and the combined organic layers are washed with brine, dried over sodium sulfate, filtered, and the solvent then removed by distillation under vacuum. The vacuum distilled mixture is purified by flash chromatography to provide a yellow oil (223 mg, 78% yield) comprising the unsaturated ester of formula VII ((2E,4E,6R)-7-[4-(dimethylamino)phenyl]-7-methoxy-4,6-dimethyl-2,4-heptadienoate) as a 2:1 mixture of trans:cis isomers. Analytical: ¹H NMR (CDCl₃) δ (trans) 7.44-7.35 (1H, d, J=15.6 Hz, —CH═CHCO₂Me), 7.18-7.10+6.77-6.70 (4H, AB, J=8.4 Hz, ArH's), 5.92-5.85 (1H, d, J=9.6 Hz, —CH═CMe—), 5.84-5.76 (1H, d, J=15.6 Hz, ═CHCO₂Me), 3.92-3.88 (1H, d, J=6.9 Hz, CHOMe), 3.78 (3H, s, CO₂Me), 3.17 (3H, s, OMe), 2.98 (6H, s, NMe₂), 2.98-2.82 (1H, m, MeOCHCH(Me)CH=), 1.73 (3H, s, ═CH(Me)CH═), 0.89-0.85 (3H, d, J=6.6 Hz, CHMe); (cis isomer key differences) 7.72-7.62 (1H, d, J=15.6 Hz, —CH═CHCO₂Me), 5.92-5.82 (1H, d, J=15.6 Hz, ═CHCO₂Me), 5.75-5.68 (1H, d, J=9.9 Hz, —CH═CMe—), 3.90-3.85 (1H, d, J undeterminable, CHOMe), 3.78 (3H, s, CO₂Me), 1.89 (3H, s, ═CH(Me)CH═); m/z (+ve FAB) 318 (M+H); HRMS for C₁₉H₂₇NO₃ +H calcd 318.2069, found 318.2047.

Step 5: Saponification of Unsaturated Ester (Formula VII) to Carboxylic Acid (Formula VIII).

The unsaturated ester of formula VII obtained in step 5 is saponified with LiOH according to the procedure described in K. Mori et al, “Synthetic Microbial Chemistry: Synthesis of Trichostatin-A, a Potent Differentiation Inducer of Friend Leukemic-Cells, and its Antipode,” Tetrahedron, 44 (1988): pp. 6013-20. Work up utilizes phosphate buffer solutions (pH 7 and 4) for an easier extraction and then column chromatography to provide a purified product (90% yield) comprising the carboxylic acid of formula VIII ((2E,4E,6R)-7-[4-(Dimethylamino)phenyl]-7-methoxy-4,6-dimethyl-2,4-heptadienoic acid). Analytical: ¹H NMR (CDCl₃) δ (major isomer) 7.49-7.41 (1H, d, J=15.6 Hz, C—CH═CHCO₂Me), 7.15-7.08+6.68-6.76 (4H, AB, J=9 Hz, ArH), 5.97-5.89 (1H, d, J=9.3 Hz, —CH═CMe—), 5.74-5.83(1H, d, J=15.9 Hz, ═CHCO₂Me), 3.85-3.92 (1H, d, J=6.9 Hz, CHOMe), 3.16 (3H, s, OMe), 2.97 (6H, s, NMe₂), 1.73 (3H, s, ═C(Me)—CH═), 0.83-0.90 (3H, d, J=6.9 Hz, —CH(Me)—).

Step 6: Synthesis of Trichostatic Acid (Formula IX).

To a stirred solution of the carboxylic acid of formula IX from step 5 (77 mg, 0.25 mmol) in DCM (1.15 mL) containing water (250 μl) is added solid 2,3-dichloro-4,5-dicyanoquinone (DDQ) (63 mg, 0.27 mmol) at room temperature. The resulting mixture is stirred for 30 min. and is then filtered through Celite. The retained solid is washed further with DCM, the combined filtrates are dried over sodium sulfate, and the solvent then removed by distillation under vacuum. The resulting crude product is vacuum distilled and is then purified by flash chromatography using a solvent gradient starting with a 1.5:98.5 mixture of isopropanol: benzene and increasing to a 4:96 mixture of isopropanol:benzene to provide a product (68 mg, 95% yield) comprising trichostatic acid of formula IX ((2E,4E,6R)-7-[4-(dimethylamino)phenyl]-4,6-dimethyl-7-oxo-2,4-heptadienoic acid) as the major component for which spectroscopic data are obtained that corresponds to the known spectroscopic data for trichostatic acid.

Step 7: Synthesis of Trichostatin A (Formula X).

The trichostatic acid from step 6 is reacted with O-(2-methoxy-2-propyl)hydroxylamine using ethyl chloroformate according to the procedure described in K. Mori et al, “Synthetic Microbial Chemistry: Synthesis of Trichostatin-A, a Potent Differentiation Inducer of Friend Leukemic-Cells, and its Antipode,” Tetrahedron, 44 (1988): pp. 6013-20 (herein incorporated by reference), to provide trichostatin A (formula X).

All documents, patents, journal articles and other materials cited in the present application are hereby incorporated by reference.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart therefrom.

What is claimed is:

1. A compound having the following formula I: 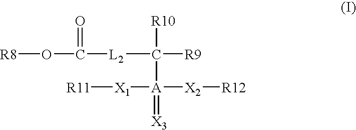
wherein R₈ is an aliphatic group, an aromatic group, or a combined aliphatic and aromatic group; R₉ and R₁₀ are each independently hydrogen, an aliphatic group, an aromatic group, a combined aliphatic and aromatic group, or R₁₀ forms a double bond with L₂ or X₃; R₁₁, and R₁₂ are each independently an aliphatic group, an aromatic group, or a combined aliphatic and aromatic group; L₂ is an aliphatic linking group, an aromatic linking group, or a combined aliphatic and aromatic linking group; A is As; L₂ being selected so that the number of carbon atoms directly in the carbon chain between the R₈ and R₉ groups is at least 4, X₁ and X₂ are each independently O or a single bond, and X₃ is O or forms R₁₃ and double bond with R₁₀, wherein R₁₃ is an aliphatic group, an aromatic group, or a combined aliphatic and aromatic group;
provided that when X₁ and X₂ are each a single bond, and when X₃ forms R₁₃ and double bond with R₁₀, A is As.

2. The compound of claim 1, wherein X₁ and X₂ are each a single bond, and X₃ forms R₁₃ and double bond with R₁₀.

3. The compound of claim 2, wherein R₁₁, R₁₂ and R₁₃ are each independently an aromatic group, or a combined aliphatic and aromatic group.

4. The compound of claim 3, wherein R₈ is methyl, and wherein R₁₁, R₁₂ and R₁₃ are each phenyl.

5. The compound of claim 1, wherein R₈ is an alkyl group having form 1 to 4 carbon atoms, R₉ is hydrogen or an alkyl group having form 1 to 4 carbon atoms, R₁₀ forms a double bond with L₂, and R₁₁ and R₁₂ are each an alkyl group having form 1 to 4 carbon atoms.

6. The compound of claim 1, wherein R₈ is a methyl group, R₉ is hydrogen or an alkyl group having form 1 to 4 carbon atoms, R₁₀ is hydrogen, an alkyl group having form 1 to 4 carbon atoms or forms a double bond with L₂, L₂ has from 4 to 5 carbon atoms and is an unsaturated aliphatic linking group or an aliphatic linking group forming a double bond with R₁₀, and R₁₁ and R₁₂ are each an ethyl group.

7. The compound of claim 6, wherein X₁, X₂ and X₃ are each O.

8. The compound of claim 6, wherein X₁ and X₂ are each a single bond, and X₃ forms R₁₃ and double bond with R₁₀.

9. The compound of claim 1, wherein L₂ is selected so that the number of carbon atoms directly in the carbon chain between the R₈ and R₉ groups is from 4 to 5.

10. The compound of claim 9, wherein L₂ is selected so that the number of carbon atoms directly in the carbon chain between the R₈ and R₉ groups is 4.

11. The compound of claim l, wherein L₂ has a double bond and/or forms a double bond with R₁₀.

12. The compound of claim 1, wherein X₁, X₂ and X₃ are each O.

13. The compound of claim 1 having the following formula II:

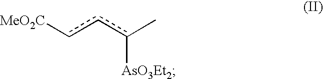

14. A process for preparing an unsaturated ester comprising the following steps:
(a) providing an aldehyde or ketone having the following formula III: 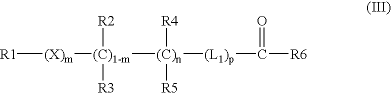
wherein R₁ is an aromatic group or a combined aliphatic and aromatic group; X is —O—, —S—, —COO—, —OOC—, —CONR₇—, or —R₇NCO—; L₁ an aliphatic linking group, an aromatic linking group, or a combined aliphatic and aromatic linking group; R₂ and R₃ are each independently hydrogen, a hydroxy group, an alkoxy group, an amino group, a carboxy group, an amide group, an ester group, a carbamate group, an aliphatic group, an aromatic group, a combined aliphatic and aromatic group, R₂ and R₃ together are ═O, or one of R₂ and R₃ form a double bond with one of R₄ and R₅; R₄ and R₅ are each independently hydrogen, a hydroxy group, an alkoxy group, an amino group, a carboxy group, an amide group, an ester group, a carbamate group, an aliphatic group, an aromatic group, a combined aliphatic and aromatic group, or one of R₄ and R₅ form a double bond with one of R₂ and R₃; R₆ is hydrogen, an aliphatic group, an aromatic group, or a combined aliphatic and aromatic group; R₇ is hydrogen, an aliphatic group, an aromatic group, or a combined aliphatic and aromatic group; m is 0 or 1; n is 0 or 1; and p is 0 or 1; m, n, p, and L₁ being selected so that the number of carbon atoms directly in the carbon chain between the R₁ and R₆ groups is at least 2;
(b) providing an ester having the following formula IV: 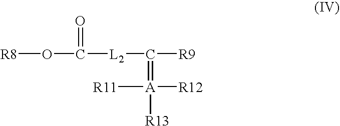
wherein R₈ is an aliphatic group, an aromatic group, or a combined aliphatic and aromatic group; R₉ is hydrogen, an aliphatic group, an aromatic group, or a combined aliphatic and aromatic group; R₁₁, R₁₂ and R₁₃ are each independently an aliphatic group, an aromatic group, or a combined aliphatic and aromatic group; L₂ is an aliphatic linking group, an aromatic linking group, or a combined aliphatic and aromatic linking group; A is P or As; L₂ being selected so that the number of carbon atoms directly in the carbon chain between the R₈ and R₉ groups is at least 4;
(c) reacting the ester of formula IV with the aldehyde or ketone of formula III to form an unsaturated ester having the following formula V:

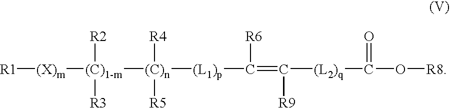

15. The process of claim 14 wherein A is P.

16. The process of claim 14 wherein A is As.

17. The process of claim 14 wherein R ₁₁, R₁₂ and R₁₃ are each independently an aromatic group, or a combined aliphatic and aromatic group.

18. The process of claim 17, wherein R₈ is methyl, and wherein R₁₁, R₁₂ and R₁₃ are each phenyl.

19. The process of claim 14, wherein step (c) is carried out in presence of a base.

20. The process of claim 19, wherein the base is sodium hydroxide, potassium hydroxide, lithium hydroxide, ammonium hydroxide, sodium methoxide, potassium methoxide, lithium methoxide, lithium diisopropylamide, sodium bis(trimethylsilyl)amide, potassium bis(trimethylsilyl)amide, sodium hydride, potassium hydride, or lithium hydride.

21. The process of claim 14, wherein R₆ is hydrogen.

22. The process of claim 21, wherein R₁ is a phenyl group, substituted phenyl group, benzyl group, quinolinyl group, or substituted quinolinyl group.

23. The process of claim 22, wherein p is 1.

24. The process of claim 23, wherein L₁ is an aromatic linking group, or a combined aliphatic and aromatic linking group.

25. The process of claim 21, wherein m is 1, wherein X is —R₇NCO—, and wherein R₇ is hydrogen.

26. The process of claim 25, wherein one of R₄ and R₅ is an amide group or carbamate group.

27. The process of claim 26, wherein one of R₄ and R₅ is an amide group —OCNHR₁₄ or a carbamate group —NHCOOR₁₄, and wherein R₁₄ is an aromatic group or a combined aromatic and aliphatic group.

28. The process of claim 21, wherein m is 0, wherein one of R₂ and R₃ is an alkoxy group having form 1 to 4 carbon atoms, and wherein the other of R₂ and R₃ is hydrogen.

29. The process of claim 28, wherein the alkoxy group is a methoxy group.

30. A process for preparing trichostatic acid or trichostatin A comprising the following steps:
(a) providing an aldehyde having the following formula VI: 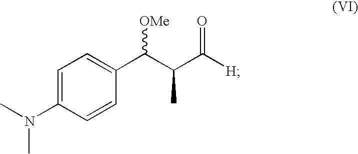
(b) providing an unsaturated ester having the following formula VII: 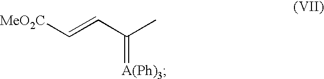
(c) reacting the aldehyde of formula VI with the unsaturated ester of formula VII to provide an unsaturated ester having the following formula VIII: 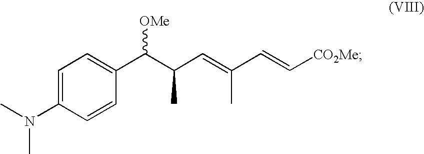
(d) hydrolyzing the unsaturated ester of formula VIII to provide a carboxylic acid having the following formula IX: 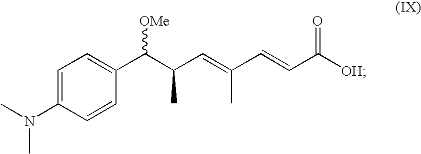
(e) converting the carboxylic acid of formula IX to trichostatic acid; and
(f) optionally converting the trichostatic acid of step (e) to trichostatin A.

31. The process of claim 30, wherein step (c) is carried out by adding a base to the unsaturated ester of formula VII before reacting with the aldehyde of formula VI.

32. The process of claim 31, wherein the base is sodium hydroxide, potassium hydroxide, lithium hydroxide or ammonium hydroxide.

33. The process of claim 30, wherein A is P.

34. The process of claim 30, wherein A is As.
